(19) United States
(12) Patent Application Publication
(10) Pub. No.: US 2024/0306957 A1
(43) Pub. Date: Sep. 19, 2024

(54) EXECUTING NON-INVASIVE RF ANALYTE MEASUREMENTS IN OPERATIVE PROCEDURES

(71) Applicant: KNOW LABS, INC., Seattle, WA (US)

(72) Inventor: JOHN CRONIN, Seattle, WA (US)

(21) Appl. No.: 18/605,358

(22) Filed: Mar. 14, 2024

Related U.S. Application Data
(60) Provisional application No. 63/490,893, filed on Mar. 17, 2023.

(51) Int. Cl. A61B 5/145; A61B 5/00; A61B 5/05; A61B 34/30

(52) U.S. Cl. CPC A61B 5/14546; A61B 5/0022; A61B 5/05; A61B 5/6824; A61B 34/30

(57) ABSTRACT

A system that includes an apparatus for generating radio frequency scanning data which includes a transmitter for transmitting radio waves below the skin surface of a person and a two-dimensional array of receive antennas for receiving the radio waves, including a reflected portion of the transmitted radio waves that is reflected from a blood vessel of the person. The wave signal is compared to known standard waveforms, and similar waveforms are input into a machine learning algorithm to determine one or more health parameters of the person. The system then notifies the person and/or health professionals of the person's health status. The apparatus may be activated by any number of modules when data is needed. The apparatus may be activated based on the requirements of medical staff, the requirements of other measurement devices, the requirements of an operating device or robot, a set schedule, etc.

### FIELD

The present disclosure is generally related to systems and methods of monitoring health parameters and, more particularly, relates to a system and a method of monitoring real-time analyte levels using radio frequency signals.

### BACKGROUND

Current medical technology fails to provide an effective solution for integrating real-time non-invasive analyte data in pre-operative, operative, and post-operative surgical care. This results in an inability to monitor and respond to changes in patient condition during surgical care, leading to an increased risk of surgical complications and decreased patient outcomes.

There is a need for a system or method that can effectively integrate real-time non-invasive analyte data in surgical care, providing healthcare practitioners with accurate and up-to-date information for improved patient care.

### SUMMARY

A system and method that can effectively integrate real-time non-invasive analyte data in surgical care, providing healthcare practitioners with accurate and up-to-date information for improved patient care.

In an embodiment, a method of executing a non-invasive RF analyte measurement in operative procedures can include providing a system for non-invasive RF analyte analysis; executing a base module to integrate operative procedures with a non-invasive RF analyte analysis device; executing at least two of integrated operative procedures with the non-invasive RF analyte analysis device, from the list including: executing prescription module that uses a doctor to prescribe obtaining real-time non-invasive RF analyte measurements by the non-invasive RF analyte device, or executing a device module that integrates the operating room with some medical equipment that are connected to the non-invasive RF analyte device, or executing a guidance module using a medical guidance rules-based engine to trigger non-invasive RF analyte measurements by the non-invasive RF analyte device, or executing a workflow module that triggers real-time non-invasive RF analyte measurements by the non-invasive RF analyte device, or executing a robot module that connects an operating room robot to the non-invasive RF analyte device, or executing an OR module that connects at least one operating room user interface to the non-invasive RF analyte device, or executing a profile module that inputs a user profile, recommends non-invasive RF analyte measurements and schedule, and data collection, or executing a wearable module that is connected to the non-invasive RF analyte device.

A surgical care patient health monitoring system can include a non-invasive device that includes one or more transmit antennas configured to transmit radio frequency (RF) analyte detection signals from the one or more transmit antennas into a patient during surgical care, and one or more receive antennas that receive return RF analyte signals that result from the transmitted RF analyte detection signals into the patient during the surgical care, the non-invasive device is configured to be triggered to perform an analyte detection in the patient using the one or more transmit antennas and the one or more receive antennas during the surgical care. The system can also include at least one of a prescription module, a device module, a guidance module, a workflow module, a robot module, an OR module, a profile module, and/or a wearable module.

The prescription module prescription module, the device module, the guidance module, the workflow module, the robot module, the OR module, the profile module, and/or the wearable module are each individually connectable to and able to control operation of the non-invasive device to perform the analyte detection in the patient during the surgical care. The prescription module is configured to allow a user to enter a prescription that is used to trigger the analyte detection by the non-invasive device during the surgical care. The device module is connectable during the surgical care to a medical device that is used during the surgical care and that is able to trigger the analyte detection by the non-invasive device via the device module during the surgical care. The guidance module is configured to search for medical guidance based on the patient during the surgical care, and the guidance module is able to trigger the analyte detection by the non-invasive device based on the medical guidance during the surgical care. The workflow module is configured to allow a user to select an existing workflow or create a new workflow during the surgical care, and the workflow module is able to trigger the analyte detection by the non-invasive device based on the existing workflow or the new workflow during the surgical care. The robot module is configured to connect during the surgical care to a surgical robot that is used during the surgical care and that is able to trigger the analyte detection by the non-invasive device via the robot module during the surgical care. The OR module is configured to allow a user to check-in to the OR module during the surgical care and allow the user to trigger the analyte detection by the non-invasive device via the OR module during the surgical care. The profile module is configured to allow a user to enter and/or retrieve patient profile information of the patient from a record or database during the surgical care, check a database for a suggested analyte measurement based on the patient profile information during the surgical care, and trigger the analyte detection by the non-invasive device during the surgical care based on the suggested analyte measurement. The wearable module is configured to connect during the surgical care to a wearable medical device that is worn by the patient during the surgical care, and configured to trigger the analyte detection by the non-invasive device during the surgical care based on data detected by the wearable medical device.

A surgical care patient health monitoring method can include providing a non-invasive device that includes one or more transmit antennas configured to transmit radio frequency (RF) analyte detection signals from the one or more transmit antennas into a patient during surgical care, and one or more receive antennas that receive return RF analyte signals that result from the transmitted RF analyte detection signals into the patient during the surgical care, the non-invasive device is configured to be triggered to perform an analyte detection in the patient using the one or more transmit antennas and the one or more receive antennas during the surgical care. The method can also include providing or executing at least one of the prescription module, the device module, the guidance module, the workflow module, the robot module, the OR module, the profile module, and/or the wearable module.

### DRAWINGS

FIG. 1: Illustrates a radio frequency health monitoring system, according to an embodiment.

FIG. 2: Illustrates an example operation of a Device Base Module, according to an embodiment.

FIG. 3: Illustrates an example operation of an Input Waveform Module, according to an embodiment.

FIG. 4: Illustrates an example operation of a Matching Module, according to an embodiment.

FIG. 5: Illustrates an example operation of a Machine Learning Module, according to an embodiment.

FIG. 6: Illustrates an example operation of a Notification Module, according to an embodiment.

FIG. 7: Illustrates an example operation of a Base Module, according to an embodiment.

FIG. 8: Illustrates an example operation of a Prescription Module, according to an embodiment.

FIG. 9: Illustrates an example operation of a Device Module, according to an embodiment.

FIG. 10: Illustrates an example operation of a Guidance Module, according to an embodiment.

FIG. 11: Illustrates an example operation of a Workflow Module, according to an embodiment.

FIG. 12: Illustrates an example operation of a Robot Module, according to an embodiment.

FIG. 13: Illustrates an example operation of an OR Module, according to an embodiment.

FIG. 14: Illustrates an example operation of a Profile Module, according to an embodiment.

FIG. 15: Illustrates an example operation of a Wearable Module, according to an embodiment.

FIG. 16: Illustrates an example of a Glucose Waveform, according to an embodiment.

FIG. 17: Illustrates an example of Matching Methods, according to an embodiment.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

U.S. Pat. Nos. 10,548,503, 11,063,373, 11,058,331, 11,033,208, 11,284,819, 11,284,820, 10,548,503, 11,234,619, 11,031,970, 11,223,383, 11,058,317, 11,193,923, 11,234,618, 11,389,091, U.S. 2021/0259571, U.S. 2022/0077918, U.S. 2022/0071527, U.S. 2022/0074870, U.S. 2022/0151553, are each individually incorporated herein by reference in their entirety.

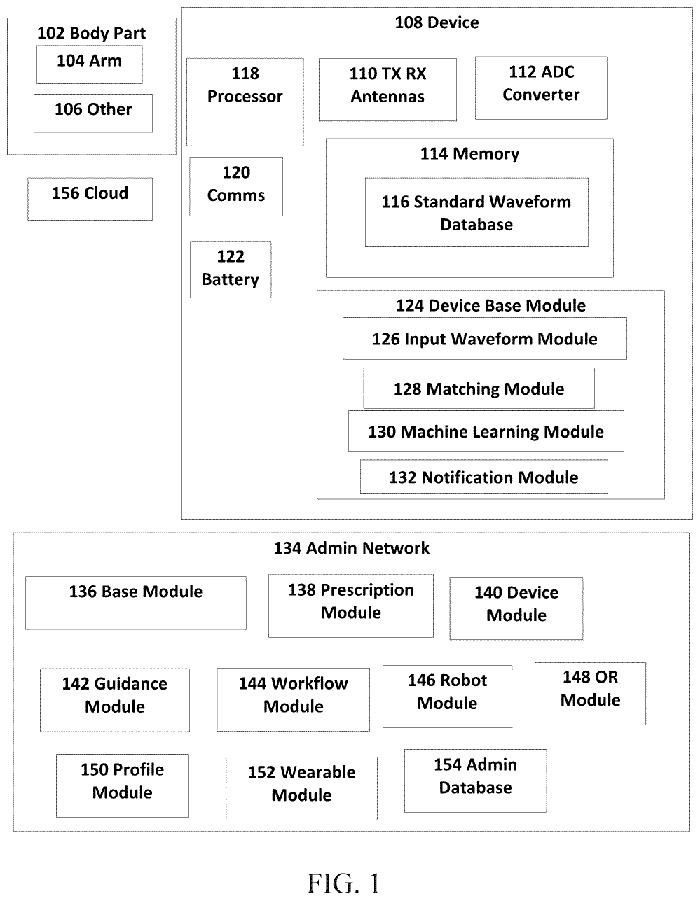

FIG. 1 illustrates a system for radio frequency health monitoring. This system comprises a body part 102, to which the device 108 is attached or in proximity to. The body part 102 may be an arm 104. The body part 102 may be the other arm of the patient or another body part 106 besides an arm, such as a leg, finger, chest, head, or any other body part from which useful medical parameters can be taken. The system may further comprise the device 108, which may be a wearable and portable device such as, but not limited to, a cell phone, a smartwatch, a tracker, a wearable monitor, a wristband, and a personal blood monitoring device.

The system may further comprise a set of TX antennas 110 and RX antennas 168. TX antennas 110 may be configured to transmit RF signals in the RF Activated Range from 500 MHZ to 300 GHZ. In one embodiment, a pre-defined frequency may correspond to a range suitable for the human body. For example, the one or more TX antennas 110 would use radio frequency signals at a range of 120-126 GHz. Successively, the one or more RX antennas 168 may be configured to receive the RF signals in response to the TX RF signal. The system may further comprise an ADC converter 112, which may be configured to convert the received RF signals from an analog signal into a digital processor readable format.

The system may further comprise memory 114, which may be configured to store the transmitted RF signals by the one or more TX antennas 110 and receive a portion of the received RF signals from the one or more RX antennas 168. Further, the memory 114 may also store the converted digital processor readable format by the ADC converter 112. The memory 114 may include suitable logic, circuitry, and/or interfaces that may be configured to store a machine code and/or a computer program with at least one code section executable by the processor 118. Examples of implementation of the memory 114 may include, but are not limited to, Random Access Memory (RAM), Read Only Memory (ROM), Hard Disk Drive (HDD), and/or a Secure Digital (SD) card.

The system may further comprise a standard waveform database 116, which may contain standard waveforms for known patterns. These may be raw or converted device readings from patients or persons with known conditions. For example, the standard waveform database 116 may include raw or converted device readings from the patient, for example the right arm, known to have diabetes or an average of multiple patients. This data can be compared to readings from a person with an unknown condition to determine if the waveforms from that person match any of the known standard waveforms.

The system may further comprise a processor 118, which may facilitate the operation of the device 108 according to the instructions stored in the memory 114. The processor 118 may include suitable logic, circuitry, interfaces, and/or code that may be configured to execute a set of instructions stored in the memory 114.

The system may further comprise comms 120, which may communicate with a network. Examples of networks may include, but are not limited to, the Internet, a cloud network, a Wireless Fidelity (Wi-Fi) network, a Wireless Local Area Network (WLAN), a Local Area Network (LAN), Long Term Evolution (LTE), and/or a Metropolitan Area Network (MAN).

The system may further comprise a battery 122, which may power hardware modules of the device 108. The device 108 may be configured with a charging port to recharge the battery 122. Charging of the battery 122 may be achieved via wired or wireless means.

The system may further comprise a device base module 124, which may be configured to store instructions for executing the computer program on the converted digital processor readable format signals of the ADC converter 112. The device base module 124 may be configured to facilitate the operation of the processor 118, the memory 114, the TX antennas 110 and RX antennas 168, and the comms 120. Further, the device base module 124 may be configured to create polling of the RF Activated Range signals from 500 MHZ to 300 GHZ. It can be noted that the device base module 124 may be configured to filter the RF Activated Range signals from 500 MHZ to 300 GHZ received from the one or more RX antennas 168.

The system may further comprise an input waveform module 126, which may extract a radio frequency waveform from memory. This may be the raw or converted data recording from the RX antennas 168 from a patient wearing the device 108. If the entire radio frequency is too long for effective matching, the input waveform module 126 may select a time interval within the data set. This input waveform may then be sent to the matching module 128.

The system may further comprise a matching module 128, which may match the input waveform and each of the standard waveforms in the standard waveform database 116 by performing a convolution and/or cross-correlation of the input waveform and the standard waveform. These convolutions and/or cross-correlations are then sent to the machine learning module.

The system may further comprise a machine learning module 130 which has been trained to identify health parameters based on the convolution and/or cross-correlations of the input and standard waveforms. The machine learning module 130 receives the convolutions and cross-correlations from the matching module 128 and outputs any health parameters identified.

The system may further comprise a notification module 132, which may determine if any of the health parameters output by the machine learning module 130 require a notification. If so, the patient and/or the patient's medical care providers may be notified.

In some embodiments, the device base module 124 may utilize a motion module 156 that includes at least one sensor from the group of an accelerometer, a gyroscope, an inertial movement sensor, or other similar sensor. The motion module 156 may have its own processor or utilize the processor 118 to calculate the user's movement. Motion from the user will change the blood volume in a given portion of their body and the blood flow rate in their circulatory system. This may cause noise, artifacts, or other errors in the real-time signals received by the RX antennas 168. The motion module 156 may compare the calculated motion to a motion threshold stored in memory 114. For example, the motion threshold could be movement of more than two centimeters in one second. The motion threshold could be near zero to ensure the user is stationary when measuring to ensure the least noise in the RF signal data. When calculated motion levels exceed the motion threshold, the motion module 156 may flag the RF signals collected at the time stamp corresponding to the motion as potentially inaccurate. In some embodiments, the motion module 156 may compare RF signal data to motion data over time to improve the accuracy of the motion threshold. The motion module 156 may alert the nurse, doctor, or other medical personel, such as with an audible beep or warning or a text message or alert to a connected mobile device. The alert would signal the nurse, doctor, or other medical personel, that the patient is moving too much to get an accurate measurement. The motion module 156 may update the standard waveform database 116 with the calculated motion of the user that corresponds with the received RF signal data. In this manner, the motion module 156 may be simplified to just collect motion data and allow the device base module 124 to determine if the amount of motion calculated exceeds a threshold that would indicate the received RF signal data is too noisy to be relied upon for a blood glucose measurement.

The device base module 124 may utilize a body temperature module 158 that includes at least one sensor from the group of a thermometer, a platinum resistance thermometer (PRT), a thermistor, a thermocouple, or another temperature sensor. The body temperature module 158 may have its own processor or utilize the processor 118 to calculate the temperature of the user or the user's environment. The user's body temperature, the environmental temperature, and the difference between the two will change the blood volume in a given part of their body and the blood flow rate in their circulatory system. Variations in temperature from the normal body temperature or room temperature may cause noise, artifacts, or other errors in the real-time signals received by the RX antennas 168. The body temperature module 158 may compare the measured temperature to a threshold temperature stored in memory 114. For example, the environmental temperature threshold may be set at zero degrees Celsius because low temperatures can cause a temporary narrowing of blood vessels which may increase the user's blood pressure. When the measured temperature exceeds the threshold, the body temperature module 158 may flag the RF signals collected at the time stamp corresponding to the temperature as potentially being inaccurate. In some embodiments, the body temperature module 158 may compare RF signal data to temperature data over time to improve the accuracy of the temperature threshold. The body temperature module 158 may alert the nurse, doctor, or other medical personel, such as with an audible beep or warning or a text message or alert to a connected mobile device. The alert would signal to the nurse, doctor, or other medical personel that the patient's body temperature, or the environmental temperature is not conducive to getting an accurate measurement. The body temperature module 158 update the standard waveform database 116 with the measured user or environmental temperature that corresponds with the received RF signal data. In this manner, the body temperature module 158 may be simplified to just collect temperature data and allow the device base module 124 to determine if the temperature measure exceeds a threshold that would indicate the received RF signal data is too noisy to be relied upon for a blood glucose measurement.

The device base module 124 may utilize an ECG module 162 that includes at least one electrocardiogram sensor. The ECG module 162 may have its own processor or utilize the processor 118 to record the electrical signals that correspond with the user's heartbeat. The user's heartbeat will impact blood flow. Measuring the ECG data may allow the received RF data to be associated with peak and minimum cardiac output so as to create a pulse waveform allowing for the estimation of blood volume at a given point in the wave of ECG data. Variations in blood volume may cause noise, artifacts, or other errors in the real-time signals received by the RX antennas 168. The ECG module 162 may compare the measured cardiac data to a threshold stored in memory 114. For example, the threshold may be a pulse above 160 bpm, as the increased blood flow volume may cause too much noise in the received RF signal data to accurately measure the blood glucose. When the ECG data exceeds the threshold, the ECG module 162 may flag the RF signals collected at the time stamp corresponding to the ECG data as potentially being inaccurate. In some embodiments, the ECG module 162 may compare RF signal data to ECG data over time to improve the accuracy of the ECG data threshold or to improve the measurement of glucose at a given point in the cycle between peak and minimum cardiac output. The ECG module 162 may alert the nurse, doctor, or other medical personel, such as with an audible beep or warning or a text message or alert to a connected mobile device. The alert would signal to the nurse, doctor, or other medical personel that the patient's heart rate is not conducive to getting an accurate measurement or requires additional medical intervention. The ECG module 162 may update the standard waveform database 116 with the measured ECG data that corresponds with the received RF signal data. In this manner, the ECG module 162 may be simplified to just collect ECG data and allow the device base module 124 to determine if the ECG data exceeded a threshold that would indicate the received RF signal data is too noisy to be relied upon for a blood glucose measurement.

The device base module 124 may include a received noise module 166 that includes at least one sensor measuring background signals such as RF signals, Wi-Fi, and other electromagnetic signals that could interfere with the signals received by the RX antennas 168. The received noise module 166 may have its own processor or utilize the processor 118 to calculate the level of background noise being received. Background noise may interfere with or cause noise, artifacts, or other errors or inaccuracies in the real-time signals received by the RX antennas 168. The received noise module 166 may compare the level and type of background noise to a threshold stored in memory 114. The threshold may be in terms of field strength (volts per meter and ampere per meter) or power density (watts per square meter). For example, the threshold may be RF radiation greater than 300 μW/m2. When the background noise data exceeds the threshold, the received noise module 166 may flag the RF signals collected at the time stamp corresponding to background noise levels as potentially being inaccurate. In some embodiments, the received noise module 166 may compare RF signal data to background noise over time to improve the accuracy of the noise thresholds. The received radiation module may alert the nurse, doctor, or other medical personel, such as with an audible beep or warning, a text message, or an alert to a connected mobile device. The alert would signal to the nurse, doctor, or other medical personel that the current level of background noise is not conducive to getting an accurate measurement. The received noise module 166 may update the standard waveform database 116 with the background noise data that corresponds with the received RF signal data. In this manner, the received noise module 166 may be simplified to just collect background noise data and allow the device base module 124 to determine if the measure exceeded a threshold that would indicate the received RF signal data is too noisy to be relied upon for a blood glucose measurement, or if an alternative transfer function should be used to compensate for the noise.

In embodiments, one or more of memory 114, standard waveform database 116, input waveform module 126, matching module 128, the machine learning module 130, the notification module 132, the motion module 156, the body temperature module 158, the ECG module 162, and/or the received noise module 166 can be provided on one or more separate devices, such as cloud server, a networked device, or the like. In such embodiments, the comms 120 can be used to communicate with the cloud server or the networked device to access the memory 114, standard waveform database 116, input waveform module 126, matching module 128, the machine learning module 130, the notification module 132, the motion module 156, the body temperature module 158, the ECG module 162, and/or the received noise module 166 by way of any suitable network.

The system may further comprise an admin network 134, which may be a computer or network of computers which receive and send information to and from the device 108 and execute one or more software modules. The admin network 134 may connect to the device 108 directly or may receive and send data over the cloud 160 or communication network.

The system may further comprise a base module 136, which may initiate the other modules of the admin network 134. The system may further comprise a prescription module 138, allowing a doctor to prescribe real-time non-invasive RF analyte measurements and activate one or more devices 108 accordingly. The system may further comprise a device module 140, which may integrate the operating room and medical devices and allow other devices to activate one or more devices 108 to collect non-invasive RF analyte measurements. Examples of medical devices include infusion pumps and cardiac equipment (ECG). The system may further comprise a guidance module 142, which may activate one or more devices 108 based on medical guidance rules. These rules may be stored locally or retrieved through the cloud 160. Medical guidance rules may activate a device 108 at various times in an operation, post-operatively, after meals, before bed, etc. The system may further comprise a workflow module 144, which may activate one or more devices 108 based on an operation room workflow. The system may further comprise a robot module 146, which may allow a surgical robot to activate one or more devices 108. A surgical robot may be a device that performs some or all of a surgical procedure. The robot may be controlled locally or remotely or may be fully autonomous. The system may further comprise an operating room (OR) module 148, which may allow operating room user interfaces to activate one or more devices 108. The system may further comprise a profile module 150, which may activate one or more devices 108 based on user profile recommendations. These recommendations may be based on medical history and/or existing conditions such as diabetes. The system may further comprise a wearable module 152, which may activate one or more devices 108 based on data from a wearable medical device. The wearable medical device may be a wearable device that is worn by the patient before, during, and/or after surgery. In some embodiments, the device 108 may activate one or more other medical devices, such as an ECG or surgical robot, based on the guidance module 142, the workflow module 144, or other medical guidance rules.

The system may further comprise an admin database 154, which may contain data used by each module of the admin network 134 and data recorded by each module of the admin network 134. The admin database 154 may be comprised of multiple databases.

The system may further comprise a Cloud 160 or communication network, which may be a wired and/or wireless network. The communication network, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art. The communication network may allow ubiquitous access to shared pools of configurable system resources and higher-level services that can be rapidly provisioned with minimal management effort, often over the Internet, and relies on sharing of resources to achieve coherence and economics of scale, like a public utility, while third-party clouds enable organizations to focus on their core businesses instead of expending resources on computer infrastructure and maintenance.

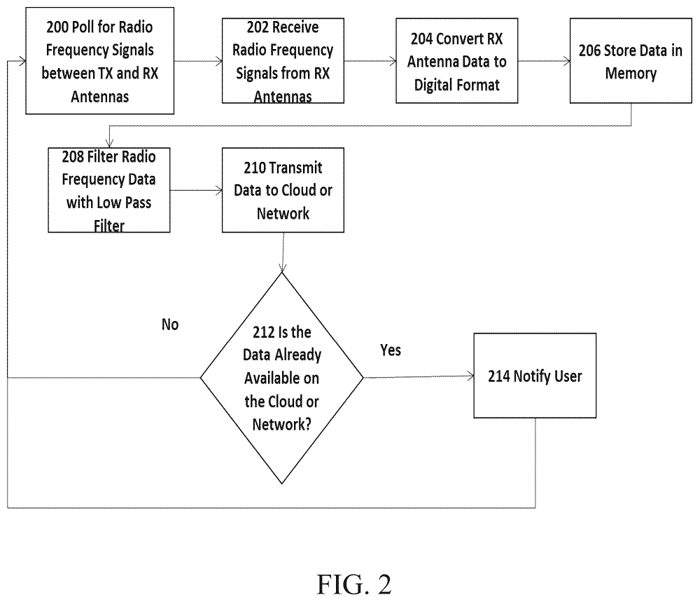

FIG. 2 illustrates an example operation of the device base module 124. The process may begin with the device base module 124 polling the Active Range RF signals between the one or more TX antennas 110 and the one or more RX antennas 168 at step 200. The device base module 124 may be configured to read and process instructions stored in the memory 114 using the processor 118. The TX antennas 110 may be configured to transmit RF signals in the RF Activated Range from 500 MHZ to 300 GHZ. For example, the one or more TX antennas 110 may use RF signals at a range of 500 MHZ to 300 GHZ. The device base module 124 may receive the RF frequency signals from the one or more RX antennas 168 at step 202. For example, an RX antenna receives an RF frequency range of 300-330 GHz from the patient's blood. The device base module 124 may be configured to convert the received RF signals into a digital format using the ADC 112 at step 204. For example, the received RF signals of frequency range 300-330 GHz is converted into a 10-bit data signal. The device base module 124 may be configured to store converted digital format signals into the memory 114 at step 206. The device base module 124 may be configured to filter the stored RF signals at step 208. The device base module 124 may be configured to filter each RF signal using a low pass filter. For example, the device base module 124 filters the RF signals of frequency range 300-330 GHz to the RF of frequency range 300-310 GHZ The device base module 124 may be configured to transmit the filtered RF signals to the cloud 160 or other network using the comms 120 at step 210. For example, the device base module 124 may be configured to transmit RF signals in the RF Activated Range from 500 MHZ to 300 GHZ to the cloud 160. The device base module 124 may be configured to determine whether the transmitted data is already available in the cloud 160 or other network at step 212. The device base module 124, using the comms 120, communicates with the cloud 160 to determine that the transmitted RF signal is already available. The device base module 124 may determine that the transmitted data is not already present in the cloud 160. The device base module 124 may be redirected back to step 200 to poll the RF signals between the one or more TX antennas 110 and the one or more RX antennas 168. For example, the device base module 124 determines that the transmitted RF in the RF Activated Range from 500 MHZ to 300 GHZ is not present in the cloud 160, and corresponding to the transmitted signal, there is no data related to the blood glucose level of the patient. The device base module 124 may determine that transmitted data is already present in the cloud 160. For example, the device base module 124 reads cloud 160 notification of the patient's blood glucose level as 110 mg/dL corresponding to an RF in the RF Activated Range from 500 MHZ to 300 GHZ. The device base module 124 may continue to step 214. The device base module 124 may notify the user via the device 108 of health information, for example, blood glucose level.

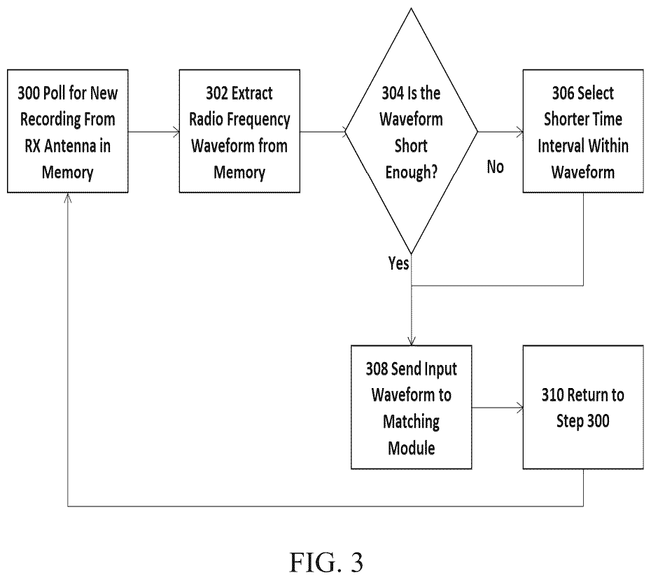

FIG. 3 illustrates an example operation of the input waveform module 126. The process may begin with the input waveform module 126 polling, at step 300, for newly recorded data from the RX antennas 168 stored in memory 114. The input waveform module 126 may extract, at step 302, the recorded radio frequency waveform from memory. If there is more than one waveform recorded, the input waveform module 126 may select each waveform separately and loop through the following steps. The input waveform module 126 may determine, at step 304, if the waveform is small enough to be an input waveform for the matching module 128. This will depend on the computational requirements and/or restrictions of the matching module 128. If the waveform is short enough, the input waveform module 126 may skip to step 308. If the waveform is too long, the input waveform module 126 may select, at step 306, a shorter time interval within the entire recorded waveform. For example, if the waveform is 5 minutes long, then only a 30-second interval may be selected. The interval may be selected at random or by a selection process. The input waveform module 126 may send, at step 308, the input waveform to the matching module 128. The input waveform module 126 may then return, at step 310, to step 300.

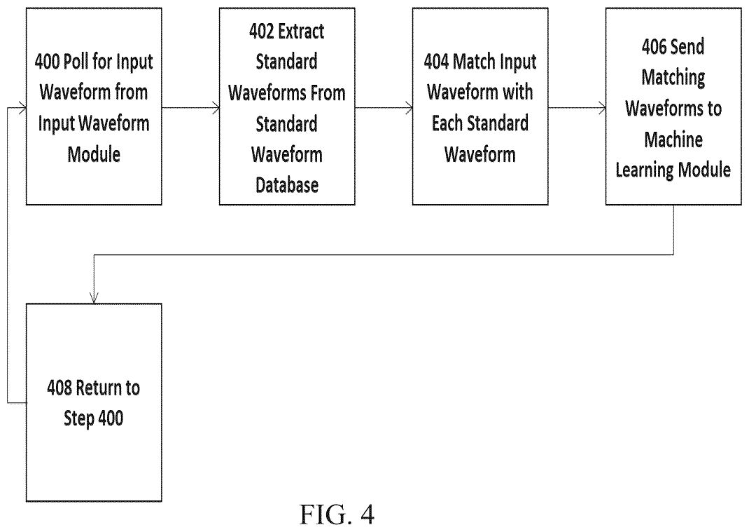

FIG. 4 illustrates an example operation of the matching module 128. The process may begin with the matching module 128 polling, at step 400, for an input waveform from the input waveform module 126. The matching module 128 may extract, at step 402, each standard waveform from the standard waveform database 116. The matching module 128 may match, at step 404, the input waveform with each standard waveform. Matching may be determining which standard waveforms the input waveform is similar to. Matching may involve convolution and/or cross-correlation of the waveforms or any other suitable matching technique. Cross-correlation and convolution are mathematical operations that can be used to determine the similarity between two wave functions. They are often used in signal processing and image recognition applications to find patterns or features in data. Cross-correlation measures the similarity between two signals as a function of the time lag applied to one of them. It is defined as the integral of the product of two signals after one is flipped and delayed by some amount. By running the cross-correlation function on two wave functions, the output will give a similarity value between two signals, where the highest value represents the most similar pair. Convolution, on the other hand, is a mathematical operation that combines two functions to produce a third function. It is the integral of the product of two functions after one of them is flipped and then shifted. By applying convolution on two wave functions, the output will be a function in which values represent the degree of similarity between input signals, where higher values represent more similar signals. These operations may be used in combination and/or with other techniques, such as Fourier transform, to extract information from signals and compare them. Matching waveforms may be waveforms where the cross-correlation and/or convolution values are close to 1 with respect to time. For example, the threshold value may be 0.85. Any point in the function that results from cross-correlation above 0.85 may indicate that the standard waveform matches the input waveform. Matching standard waveforms, the input waveform, the cross-correlation of both, and/or the convolution of both may be used as an input to the machine learning algorithm of the machine learning module 130. The matching module 128 may send, at step 406, the matching waveforms to the machine learning module 130. Matching waveforms may refer to the standard waveforms that were similar to the input waveform, the waveforms that were generated via convolution and/or cross-correlation, or both. The matching module 128 may then return, at step 408, to step 400.

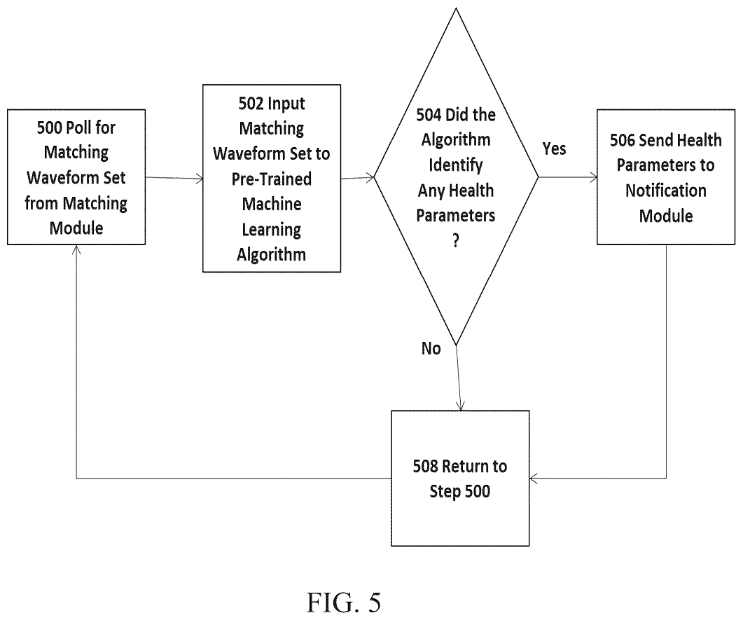

FIG. 5 illustrates an example operation of the machine learning module 130. The process may begin with the machine learning module 130 polling, at step 500, for a set of matching waveforms from the matching module 128. Matching waveforms may be a set of standard waveforms that are similar to the input waveform or statistical combinations of the input waveform and standard waveforms, such as convolutions or cross-correlations. The machine learning module 130 may input, at step 502, the set of received waveforms into a pre-trained machine learning algorithm. The machine learning algorithm may be trained on similar sets of matched waveforms where the input waveform is from a patient whose health parameters are known. The waveforms may be input directly into the algorithm, such as a set of X and Y values. The matching waveforms may each be summarized as a closest fit function or may be transformed into a set of sine waves using a Fourier transform. Training data should be labeled with the correct output, such as the type of waveform. In order to prepare the data, the waveforms need to be processed and converted into a format that can be used by the algorithm. Once the data is prepared, the algorithm is trained on the labeled data. The model uses this data to learn the relationships between the waveforms and their corresponding outputs. During training, the model will adjust its parameters to minimize errors between its predictions and the correct outputs. Once the model has been trained and fine-tuned, it can recognize waveforms in new, unseen data. This could be done by giving the input waveforms, then the algorithm will predict the health parameters. The machine learning module 130 may determine, at step 504, if the algorithm identified any health parameters. Identification may require a certain interval of confidence. For example, if the machine learning algorithm determines that it is more than 70% likely that a health parameter is correct, then that parameter may be considered identified. If multiple conflicting parameters exist, then the most confident may be used. For example, if the algorithm determines that it is 75% likely that the patient's blood glucose level is between 110-115 mg/dL and 90% likely that the patient's blood glucose is between 105-110 mg/dL, then the more confident value of 105-110 mg/dL may be identified. If none of the results from the machine learning algorithm are above the confidence threshold, or the results are otherwise inconclusive, the machine learning module 130 may skip to step 508. If any health parameters were identified, the machine learning module 130 may send, at step 506, the health parameters to the notification module 132 and/or admin network 134. The machine learning module 130 may then return, at step 508, to step 500.

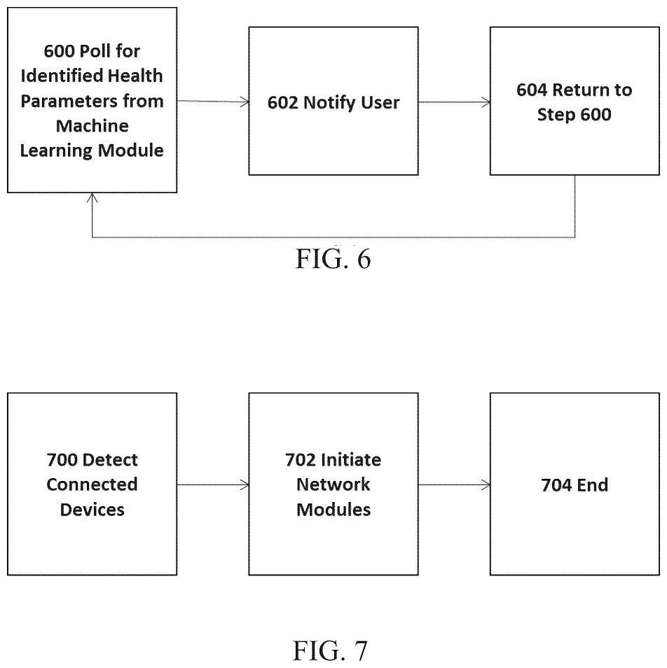

FIG. 6 illustrates an example operation of the notification module 132. The process may begin with the notification module 132 polling, at step 600, for health parameters identified by the machine learning module 130. The notification module 132 may notify, at step 602, the user of the device and/or their care providers. For example, the device may display a readable interface with the identified health parameters such as heart rate, blood pressure, blood glucose, oxygen level, etc. This information may be sent via the comms 120 to another device, such as a terminal in a nursing station, doctor's office, emergency medical transport office, etc. Notification may include audio or haptic feedback such as beeping or vibrating. The notification module 132 may then return, at step 604, to step 600.

FIG. 7 illustrates an example operation of the base module 136. The process may begin with the base module 136 detecting, at step 700, a connected device 108 or devices 108. The base module 136 may also be the module responsible for connecting devices 108 to the admin network 134. The base module 136 may initiate, at step 702, the other modules of the admin network 134, such as the prescription module 138, device module 140, the guidance module 142, workflow module 144, robot module 146, OR module 148, profile module 150, and wearable module 152. For example, the prescription module 138 may allow a doctor to prescribe a blood glucose recording from an RF device 108. For another example, the robot module 146 may allow a surgical robot to activate an RF device 108 to obtain real-time blood sodium levels. The base module 136 may then end at step 704.

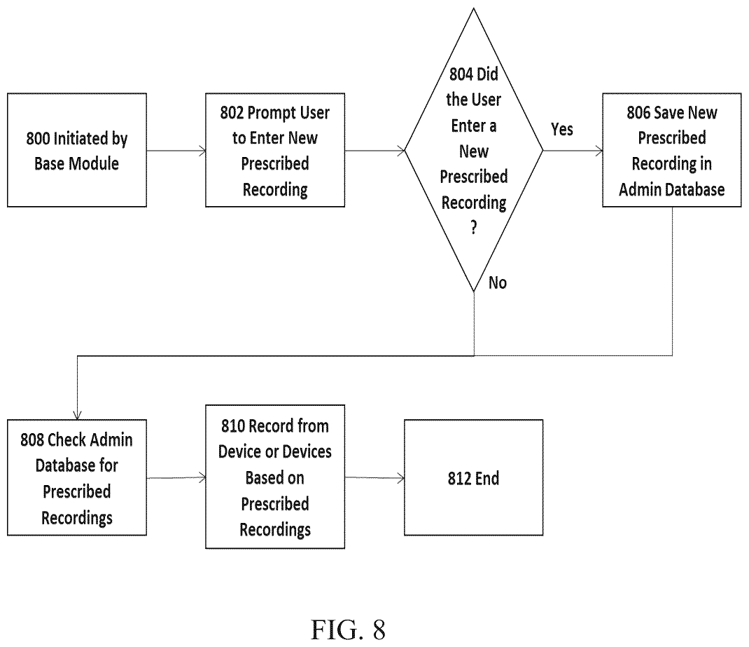

FIG. 8. illustrates an example operation of the prescription module 138. The process may begin with the prescription module 138 being initiated, at step 800, by the base module 136. The prescription module 138 may prompt, at step 802, a user to enter a new prescribed recording. Users may refer to a doctor, other health professionals, or anyone authorized to prescribe a recording from a device 108. The prescription may include the analyte or analytes to be measured, the time and duration of the recording, the identity of the patient, etc. The prescription module 138 may determine, at step 804, if the user entered a new prescribed recording. If not, the prescription module 138 may skip to step 808. If the user enters a new prescribed recording, the prescription module 138 may save, at step 806, the prescribed recording in the admin database 154. The prescription module 138 may check, at step 808, the admin database 154 for prescribed recordings. Some prescriptions may be removed from the admin database 154 after the recording is made, while others may be recurring. The prescription module 138 may record, at step 810, analyte level data from a device 108 or devices 108. The device 108 or devices 108 may be activated if they are not already. The recording may be saved in the admin database 154 and/or sent to the user or persons the user designates. The prescription module 138 may end at step 812. The prescription module 138 may not end until all prescribed recordings have been completed.

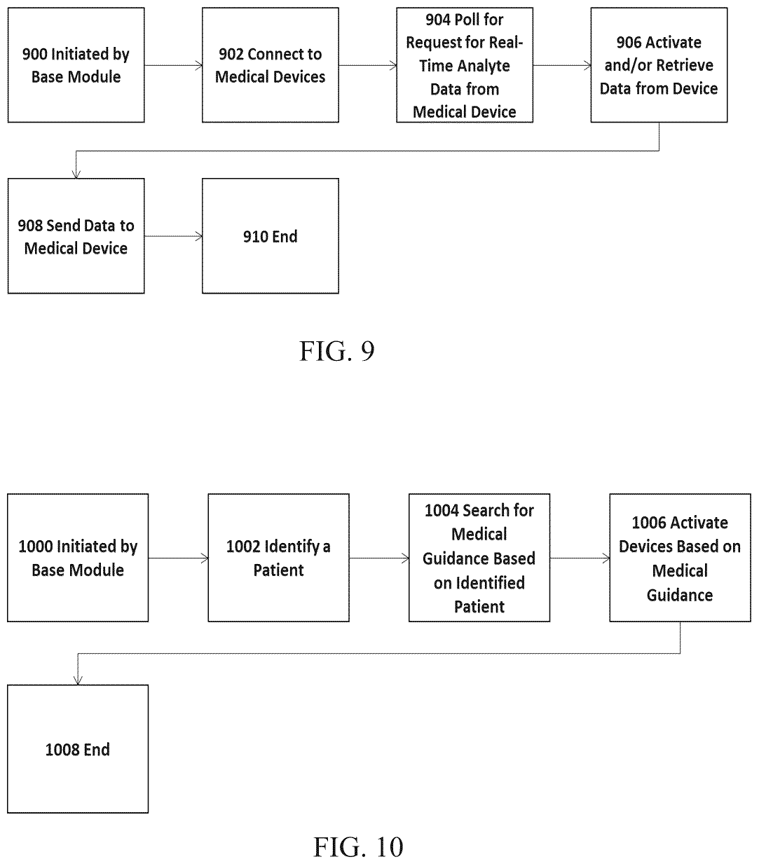

FIG. 9 illustrates an example operation of the device module 140. The process may begin with the device module 140 being initiated, at step 900, by the base module 136. The device module 140 may connect, at step 902, to any medical device. The device module 140 may identify medical devices in a selected operating room. The medical devices may need to initiate connection automatically or manually. For example, hospital staff may manually connect devices to the admin network 134 via a physical or wireless connection. Examples of medical devices may include heart rate monitors, ECGs, infusion pumps, respirators, etc. The device 108 may be a medical device, but the medical device does necessarily include the device 108. The device module 140 may poll, at step 904, for a request from one of the connected medical devices for real-time analyte data. The device module 140 may retrieve, at step 906, the real-time analyte data from one or more devices 108. The device module 140 may activate one or more devices 108. The device module 140 may send, at step 908, the requested data to the medical device. The device module 140 may end at step 910. The device module 140 may continue to run until all connected medical devices are disconnected

FIG. 10 illustrates an example operation of the guidance module 142. The process may begin with the guidance module 142 being initiated, at step 1000, by the base module 136. The guidance module 142 may identify, at step 1002, a patient. This may be the patient currently undergoing surgery in a specified operating room or manually selected by a user of the system. The guidance module 142 may select a patient from a list, such as a list of patients at a hospital or a list of patients scheduled for surgery at the current time. The guidance module 142 may search, at step 1004, for medical guidance based on the patient. The medical guidance may be based on the patient's diagnosis, symptoms, current status, vitals, etc. For example, if the patient is diabetic, the guidance module 142 may search for medical guidance on which surgical complications can arise from diabetes and which analytes should be monitored to avoid those complications. The medical guidance may refer to specific directions such as a workflow or general information such as research papers and statistics. The guidance module 142 may search the admin network 134 or an external database or databases. The guidance module 142 may be able to synthesize medical guidance from multiple sources using AI language processing. The guidance module 142 may activate, at step 1006, one or more devices 108 to record analytes based on the medical guidance. The guidance module 142 may retrieve data from these devices. The guidance module 142 may end at step 1008.

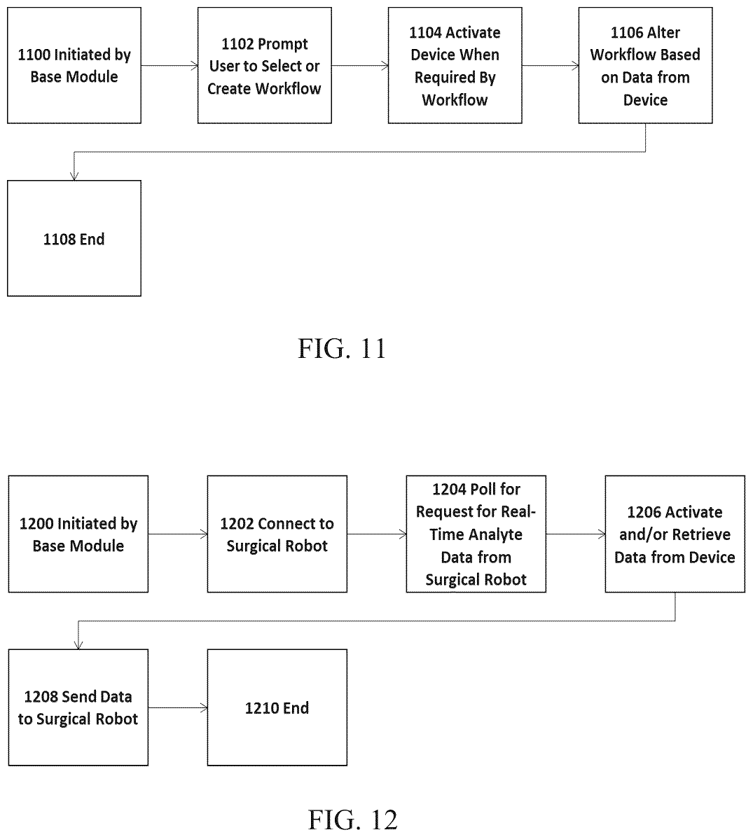

FIG. 11 illustrates an example operation of the workflow module 144. The process may begin with the workflow module 144 being initiated, at step 1100, by the base module 136. The workflow module 144 may prompt, at step 1102, a user to select an existing workflow or create a new workflow. If the user selects an existing workflow, they may also be able to edit it. A user, which may be a doctor or other medical staff, may select a workflow at the beginning of a procedure such as surgery or intake. The workflow module 144 may automatically select or generate a workflow based on information such as a surgery schedule. The workflow module 144 may activate, at step 1104, one or more devices 108 when required by the workflow. For example, if a surgical workflow involves a pre-surgery glucose level check, a device 108 may be activated to take real-time glucose measurements. The workflow module 144 may alter, at step 1106, the workflow based on the data from the activated device 108 or devices 108 if necessary. For example, the workflow may fork into different next steps based on the patient's blood glucose levels detected by the device 108. If the patient's blood glucose is above safe levels for surgery, the workflow may move to steps that would reduce the patient's glucose level instead of ending the workflow. The workflow module 144 may end at step 1108. The workflow module 144 may not end until the current workflow is completed.

FIG. 12 illustrates an example operation of the robot module 146. The process may begin with the robot module 146 being initiated, at step 1200, by the base module 136. The robot module 146 may connect, at step 1202, to a surgical robot. The robot module 146 may identify a surgical robot in a selected operating room. The surgical robot may need to initiate the connection, which may require manual assistance. For example, hospital staff may manually connect a surgical robot to the admin network 134 via a physical or wireless connection. A surgical robot may be a device that performs some or all of a surgical procedure. The robot may be controlled locally or remotely or may be fully autonomous. The robot module 146 may poll, at step 1204, for a request from the connected surgical robot for real-time analyte data. The robot module 146 may retrieve, at step 1206, the real-time analyte data from one or more devices 108. The robot module 146 may activate one or more devices 108. The robot module 146 may send, at step 1208, the requested data to the surgical robot. The robot module 146 may end at step 1210. The robot module 146 may continue to run until the surgical robot disconnects or indicates that the robot module 146 may end.

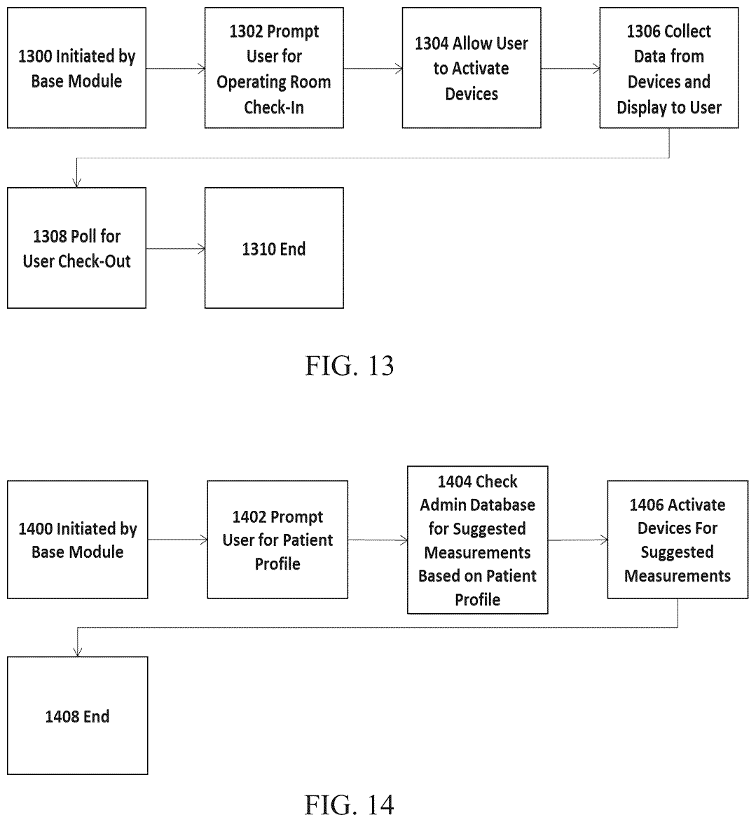

FIG. 13 illustrates an example operation of the OR module 148. The process may begin with the OR module 148 being initiated, at step 1300, by the base module 136. The OR module 148 may prompt, at step 1302, a user to check in at an operating room. Users may refer to a doctor or other medical professional. Check-in may be done through a terminal connected to, or part of, the admin network 134. Check-in may require credentials such as an ID and password. The OR module 148 may allow, at step 1304, the user to activate a device 108 or devices 108. The OR module 148 may allow the user to select which analytes to measure if the device 108 is capable of multiple analyte measurements. The OR module 148 may collect, at step 1306, data from the activated device 108 or devices 108 and display the data to the user via the check-in terminal or another display. The OR module 148 may poll, at step 1308, for the user to check out or terminate the OR module 148. The OR module 148 may end at step 1310.

FIG. 14 illustrates an example operation of the profile module 150. The process may begin with the profile module 150 being initiated, at step 1400, by the base module 136. The profile module 150 may prompt, at step 1402, a user for a patient profile. A user may refer to a doctor or other medical professional. A patient profile may include demographic information such as age, sex, gender, and ethnicity; diagnoses such as diabetes, COPD, or pneumonia; symptoms such as shortness of breath and arrhythmia; and/or any other medically relevant information. The user may enter profile information directly and/or retrieve patient information from a record or database. The profile module 150 may check at step 1404, the admin database 154 for suggested analyte measurements based on the patient's profile. For example, diabetes may be associated with a suggestion to measure blood glucose, whereas a patient presenting symptoms of bruising may be associated with a suggestion to measure hematocrit, hemoglobin, and red blood cell count (RBC). The profile module 150 may activate, at step 1406, a device 108 or devices 108 to record the suggested measurements. The profile module 150 may require user approval before the devices 108 are activated, and the user may add additional analytes to measure. The profile module 150 may end at step 1408.

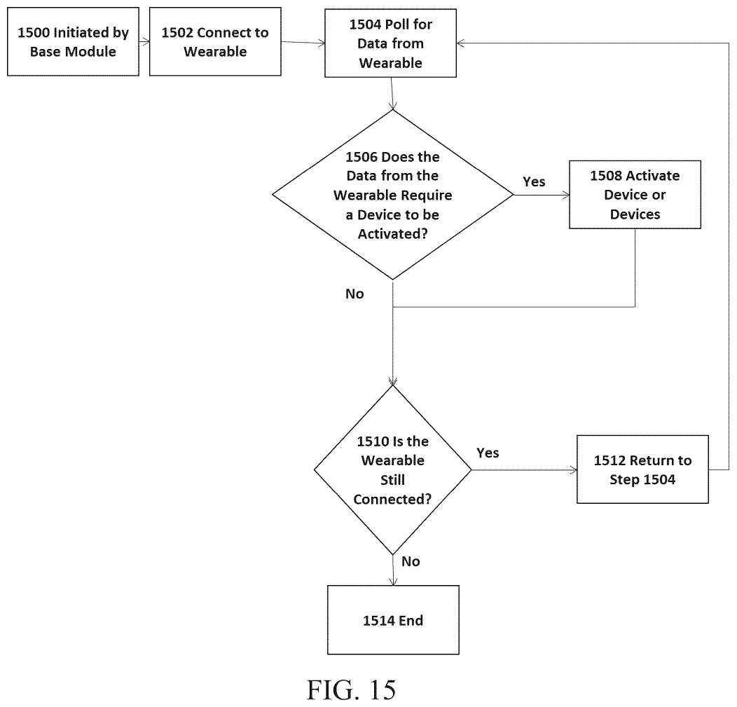

FIG. 15 illustrates an example operation of the wearable module 152. The process may begin with the wearable module 152 being initiated, at step 1500, by the base module 136. The wearable module 152 may connect, at step 1502, to a wearable medical device. The wearable medical device may be a wearable device that is worn by the patient before, during, and/or after surgery. The wearable module 152 may poll, at step 1504, for data from the wearable medical device. Examples of data from the wearable medical device may include heart rate, mobility or gait data, blood pressure, body temperature, and 3D position data. The wearable module 152 may determine, at step 1506, if the data from the wearable medical device requires a device 108 or devices 108 to be activated. This may be a direct request from the wearable medical device if capable, or the wearable module may compare the data from the wearable medical device to a set of rules in the admin database 154. If the data from the wearable medical device requires a device 108 or devices 108 to be activated, the wearable module 152 may activate, at step 1508, that device 108 or those devices 108. For example, when the heart rate is high, a device 108 may be activated to measure glucose, cortisol, and/or sodium levels. For example, when body temperature is low, a device 108 may be activated to measure thyroid-stimulating hormone (TSH) to check for hypothyroidism. If the medical wearable device data requires no device 108 to be activated, the wearable module 152 may skip to step 1510. The wearable module 152 may determine, at step 1510, if the medical wearable device is still connected. If the medical wearable device is still connected, the wearable module 152 may return, at step 1512, to step 1504. If the medical wearable device is disconnected, the wearable module 152 may end at step 1514.

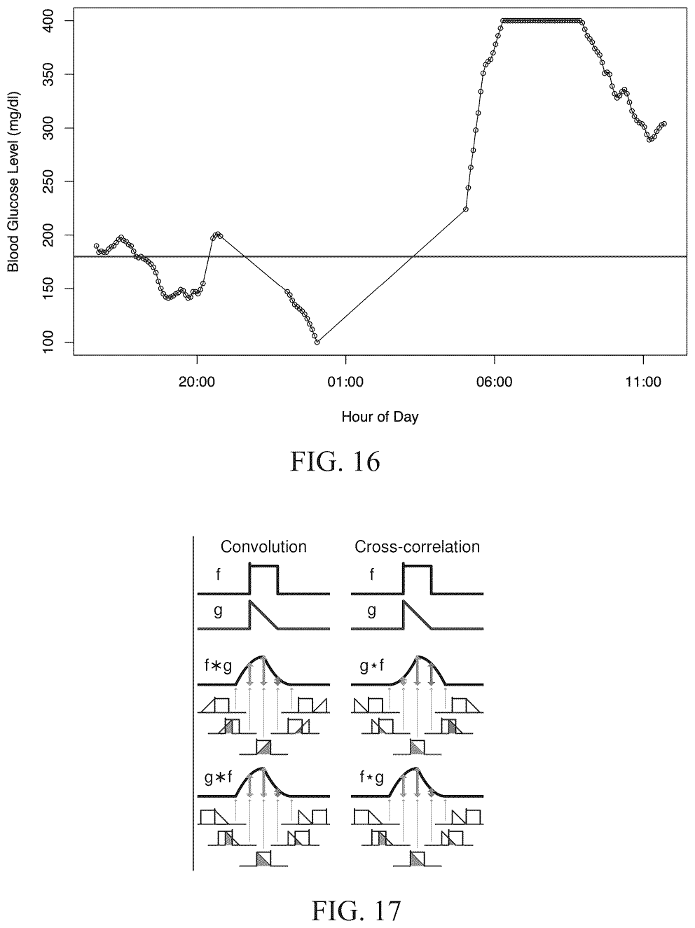

FIG. 16 displays an example of a glucose waveform. The figure shows blood glucose levels in a patient recorded over time. A computer can store a waveform by digitizing the analog signal and storing the resulting digital values in memory. Digitization is typically accomplished by an analog-to-digital converter (ADC), which samples the amplitude of the analog signal at regular intervals and converts each sample to a digital value. The resulting digital values and information about the sampling rate and bit depth can be used to reconstruct the original waveform when the data is played back. The digital values could be stored in an array or binary files. The computer may store the important parts of the waveform, such as local and/or absolute maxima and minima, inflection points, inversion points, average value, best fit line or function, etc.

FIG. 17 displays an example of matching methods such as convolution and cross-correlation. The figure illustrates two different matching methods, convolution, and cross-correlation. In the convolution process, the standard waveform slides over the input waveform, element-wise multiplying and summing the overlapping values. The result is a new output waveform. The convolution operation is useful for detecting specific features, such as edges, in the input waveform. In the cross-correlation process, the standard waveform is also sliding over the input waveform, element-wise multiplying and summing the overlapping values. However, the output waveform is not generated by summing the product of the standard waveform and the overlapping part of the input waveform but by taking the dot product of the standard waveform and the input waveform. The cross-correlation operation is used to find patterns in the input waveform that are similar to the standard waveform. Convolution and cross-correlation are similar operations used for waveform processing and pattern recognition. They are widely used in image processing, machine learning, computer vision, and waveform processing applications. This is a general description; these methods' actual implementation will depend on the specific use case and application.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

1. A surgical care patient health monitoring system, comprising:
a non-invasive device that includes one or more transmit antennas configured to transmit radio frequency (RF) analyte detection signals from the one or more transmit antennas into a patient during surgical care, and one or more receive antennas that receive return RF analyte signals that result from the transmitted RF analyte detection signals into the patient during the surgical care, the non-invasive device is configured to be triggered to perform an analyte detection in the patient using the one or more transmit antennas and the one or more receive antennas during the surgical care;
at least one of the following:
(a) a prescription module that is connectable to and able to control operation of the non-invasive device to perform the analyte detection in the patient during the surgical care; the prescription module is configured to allow a user to enter a prescription that is used to trigger the analyte detection by the non-invasive device during the surgical care;
(b) a device module that is connectable to and able to control operation of the non-invasive device to perform the analyte detection in the patient during the surgical care; the device module is connectable during the surgical care to a medical device that is used during the surgical care and that is able to trigger the analyte detection by the non-invasive device via the device module during the surgical care;
(c) a guidance module that is connectable to and able to control operation of the non-invasive device to perform the analyte detection in the patient during the surgical care; the guidance module is configured to search for medical guidance based on the patient during the surgical care, and the guidance module is able to trigger the analyte detection by the non-invasive device based on the medical guidance during the surgical care;
(d) a workflow module that is connectable to and able to control operation of the non-invasive device to perform the analyte detection in the patient during the surgical care; the workflow module is configured to allow a user to select an existing workflow or create a new workflow during the surgical care, and the workflow module is able to trigger the analyte detection by the non-invasive device based on the existing workflow or the new workflow during the surgical care;
(e) a robot module that is connectable to and able to control operation of the non-invasive device to perform the analyte detection in the patient during the surgical care; the robot module is configured to connect during the surgical care to a surgical robot that is used during the surgical care and that is able to trigger the analyte detection by the non-invasive device via the robot module during the surgical care;
(f) an OR module that is connectable to and able to control operation of the non-invasive device to perform the analyte detection in the patient during the surgical care; the OR module is configured to allow a user to check-in to the OR module during the surgical care and allow the user to trigger the analyte detection by the non-invasive device via the OR module during the surgical care;
(g) a profile module that is connectable to and able to control operation of the non-invasive device to perform the analyte detection in the patient during the surgical care; the profile module is configured to allow a user to enter and/or retrieve patient profile information of the patient from a record or database during the surgical care, check a database for a suggested analyte measurement based on the patient profile information during the surgical care, and trigger the analyte detection by the non-invasive device during the surgical care based on the suggested analyte measurement;
(h) a wearable module that is connectable to and able to control operation of the non-invasive device to perform the analyte detection in the patient during the surgical care; the wearable module is configured to connect during the surgical care to a wearable medical device that is worn by the patient during the surgical care, and configured to trigger the analyte detection by the non-invasive device during the surgical care based on data detected by the wearable medical device.

2. The surgical care patient health monitoring system of claim 1, comprising two or more of (a)-(h).

3. The surgical care patient health monitoring system of claim 1, comprising three or more of (a)-(h).

4. The surgical care patient health monitoring system of claim 1, further comprising an analog-to-digital converter connected to the one or more receive antennas.

5. The surgical care patient health monitoring system of claim 1, wherein the at least one of (a)-(h) is physically separate from the non-invasive device.

6. The surgical care patient health monitoring system of claim 2, wherein the two or more of (a)-(h) are together in an admin network.

7. A surgical care patient health monitoring method, comprising:
providing a non-invasive device that includes one or more transmit antennas configured to transmit radio frequency (RF) analyte detection signals from the one or more transmit antennas into a patient during surgical care, and one or more receive antennas that receive return RF analyte signals that result from the transmitted RF analyte detection signals into the patient during the surgical care, the non-invasive device is configured to be triggered to perform an analyte detection in the patient using the one or more transmit antennas and the one or more receive antennas during the surgical care;
at least one of the following:
(a) providing a prescription module that is connectable to and able to control operation of the non-invasive device to perform the analyte detection in the patient during the surgical care; the prescription module is configured to allow a user to enter a prescription that is used to trigger the analyte detection by the non-invasive device during the surgical care;
(b) providing a device module that is connectable to and able to control operation of the non-invasive device to perform the analyte detection in the patient during the surgical care; the device module is connectable during the surgical care to a medical device that is used during the surgical care and that is able to trigger the analyte detection by the non-invasive device via the device module during the surgical care;
(c) providing a guidance module that is connectable to and able to control operation of the non-invasive device to perform the analyte detection in the patient during the surgical care; the guidance module is configured to search for medical guidance based on the patient during the surgical care, and the guidance module is able to trigger the analyte detection by the non-invasive device based on the medical guidance during the surgical care;
(d) providing a workflow module that is connectable to and able to control operation of the non-invasive device to perform the analyte detection in the patient during the surgical care; the workflow module is configured to allow a user to select an existing workflow or create a new workflow during the surgical care, and the workflow module is able to trigger the analyte detection by the non-invasive device based on the existing workflow or the new workflow during the surgical care;
(e) providing a robot module that is connectable to and able to control operation of the non-invasive device to perform the analyte detection in the patient during the surgical care; the robot module is configured to connect during the surgical care to a surgical robot that is used during the surgical care and that is able to trigger the analyte detection by the non-invasive device via the robot module during the surgical care;
(f) providing an OR module that is connectable to and able to control operation of the non-invasive device to perform the analyte detection in the patient during the surgical care; the OR module is configured to allow a user to check-in to the OR module during the surgical care and allow the user to trigger the analyte detection by the non-invasive device via the OR module during the surgical care;
(g) providing a profile module that is connectable to and able to control operation of the non-invasive device to perform the analyte detection in the patient during the surgical care; the profile module is configured to allow a user to enter and/or retrieve patient profile information of the patient from a record or database during the surgical care, check a database for a suggested analyte measurement based on the patient profile information during the surgical care, and trigger the analyte detection by the non-invasive device during the surgical care based on the suggested analyte measurement;
(h) providing a wearable module that is connectable to and able to control operation of the non-invasive device to perform the analyte detection in the patient during the surgical care; the wearable module is configured to connect during the surgical care to a wearable medical device that is worn by the patient during the surgical care, and configured to trigger the analyte detection by the non-invasive device during the surgical care based on data detected by the wearable medical device.

8. The surgical care patient health monitoring method of claim 7, comprising two or more of (a)-(h).

9. The surgical care patient health monitoring method of claim 7, comprising three or more of (a)-(h).

10. The surgical care patient health monitoring method of claim 7, further comprising converting the received return RF analyte signals to digital signals.

11. The surgical care patient health monitoring method of claim 7, comprising arranging the at least one of (a)-(h) so that it is physically separate from the non-invasive device.

12. The surgical care patient health monitoring method of claim 8, comprising arranging the two or more of (a)-(h) so that they are together in an admin network.
